(19) United States
(12) Patent Application Publication
(10) Pub. No.: US 2013/0144052 A1
(43) Pub. Date: Jun. 6, 2013

(54) DEVICE FOR THE SYNTHESIS OF RADIO-LABELED COMPOUNDS

(71) Applicant: Marco Mueller, Radeberg (DE)

(72) Inventor: Marco Mueller, Radeberg (DE)

(73) Assignee: ABX ADVANCED BIOCHEMICAL COMPOUNDS GMBH, Radeberg (DE)

(21) Appl. No.: 13/809,506

(22) PCT Filed: Jul. 5, 2011
(86) PCT No.: PCT/DE2011/075157

§ 371 Date: Feb. 22, 2013

(30) Foreign Application Priority Data
Jul. 12, 2010 (DE) 10 2010 036 356.1

(51) Int. Cl. C07B 59/00; C07H 5/02

(52) U.S. Cl. CPC C07B 59/005; C07H 5/02
USPC 536/122; 422/159

(57) ABSTRACT

The invention relates to a device for the synthesis of radio-labeled compounds, which comprises

a reaction vessel for reacting a precursor compound having protective groups with a radioactive isotope to obtain a first reaction product;
a first cartridge for hydrolyzing the protective groups of the first reaction product to obtain a second reaction product; and
a second cartridge for purifying the second reaction product,
wherein the reaction vessel, the first cartridge, and the second cartridge are connected to each other via pipelines. Here it is provided that the first cartridge contains 801 to 1200 mg of a solid carrier and/or the reaction vessel is a reaction vessel made of a temperature-resistant plastic with the plastic having a temperature resistance of at least 120° C.

The invention relates to a device for the synthesis of radio-labeled compounds as well as the use of said device.

In the medical diagnostics there are increasingly used short-lived, radio-labeled compounds, so-called radiotracers, the physiological and biochemical properties of which enable a non-invasive tomographic detection of metabolic processes in the human body. By using the modern tomographic method of positron emission tomography (PET) metabolic processes can be quantified by means of said radiotracers and the biodistribution of the radiodiagnostic agent can be detected from the outside. The tomographic detection of radiotracers, such as for example 2-desoxy-2-[¹⁸F]fluoro-D-glucose ([¹⁸F]-FDG), allows an early diagnosis of tumors which significantly differ with respect to the glucose metabolism of normal tissue. By the development of novel radiotracers on the basis of pharmacologically interesting compounds new possibilities of the non-invasive diagnostics of various clinical pictures have opened up in the last years.

The global share of the positron emission tomography (PET) in the overall market of diagnosis by means of imaging methods has explosively increased in the last years. Here, the largest share has the [¹⁸F] fluoride as radioactive probe because in the form of the F-18 labeled sugar derivative ([¹⁸F]-FDG) it visualizes by means of PET the exact localization of tumors down to the millimeters and enables an exact localization of the tumor extension.

In general, the [¹⁸F] fluoride prepared in the cyclotron is separated from the target water by ion exchange on an anion exchange cartridge wherein as the phase transfer reagent there is often used a mixture of Kryptofix® (K2.2.2) and potassium carbonate in water/acetonitrile. Following azeotropic distillation in the subsequent synthesis step the [¹⁸F] fluoride activated by means of phase transfer catalysts is reacted with the corresponding educt (also referred to as precursor compound or precursor) in an organic solvent e.g., acetonitrile (labeling). All of the physico-chemical processes take place in synthesis modules which conditional on a number of reaction steps (e.g., ion exchange, distillation, drying, reaction) are provided with relatively complex control systems.

From DE 697 32 599 T2 there is known a device for the automated synthesis of radio-labeled compounds which should be particularly useful for the synthesis of 2-[¹⁸F]fluoro-2-desoxy-D-glucose. The device can be implemented as a so-called disposable set of equipment that is integrated in a synthesis module. In this set of equipment the reaction vessel, a first cartridge, and a second cartridge are connected to each other via pipelines. The cartridges are filled with carrier material for separating hydrophilic or lipophilic constituents of the precursor compound. On the first solid carrier after reaction of the precursor compound in the reaction vessel with the radioactive isotope the precursor compound is adsorbed on a C18 cartridge to permit the removal of protective groups by basic or acidic hydrolysis. For that, hydrochloric acid or sodium hydroxide solution is passed over the cartridge and hold on the cartridge for a longer residence time by shut-off with the help of a valve for complete hydrolysis. Among others, the cartridge can be of the C18, C18 ec, C8, C4, tC18, diol, phenyl, NH₂ type. Here, the cartridge can contain between 50 mg and 10 g of solid carrier with 200 to 800 mg being preferred. In the only example of DE 697 32 599 T2 a C18 cartridge of the Sep-Pak-Short Body type is employed for the hydrolysis that contains 400 mg of solid carrier. The reaction of the precursor compound is performed in a reaction vessel at a temperature of 105° C. Due to said high reaction temperature in contrast to the remaining vessels, pipelines, and valves the reaction vessel is not made of plastic but of glass.

With the device shown in the example of DE 697 32 599 T2 a radiochemical yield of approx. 60% can be achieved in the synthesis for [¹⁸F]-FDG. Thus, depending on the respective transport routes to the individual hospitals with an initial activity of 150 GBq there can be obtained approximately 50 patient doses with 300 MBq each.

Here, by a dose there is understood an amount of [¹⁸F]-FDG which has to be administered to a patient for a PET examination.

However, in view of the high instrumentation expenditure and the material costs for the preparation of the radioactive [¹⁸F]-FDG it is desired to significantly increase the number of doses but without increasing the engineering effort and thus, in turn the costs. However, the opportunities for optimizing the above-mentioned automated synthesis and the required device seemed to be exhausted after the many years of practical application.

It is the object of the invention to eliminate the drawbacks of the prior art. There is provided a device for the synthesis of radio-labeled compounds, in particular for the synthesis of [¹⁸F]-FDG, which enables the preparation of higher numbers of doses based on the amount of radioactive isotope used. Further, a use of said device is provided.

This object is solved by the features of claims 1, 9, and 10. Practical developments of the invention result from the features of claims 2 to 8 and 11.

In accordance to the invention a device for the synthesis of radio-labeled compounds is provided, which comprises
a reaction vessel for reacting a precursor compound having protective groups with a radioactive isotope to obtain a first reaction product;
a first cartridge for hydrolyzing the protective groups of the first reaction product to obtain a second reaction product; and
a second cartridge for purifying the second reaction product,
wherein the reaction vessel, the first cartridge, and the second cartridge are connected to each other via pipelines, and wherein the first cartridge contains 801 to 1200 mg of a solid carrier and/or the reaction vessel is a reaction vessel made of a temperature-resistant plastic with the plastic having a temperature resistance of at least 120° C.

It has been found that the increase of the amount of solid carrier on the first cartridge can already significantly increase the yield of a radio-labeled compound. The cause of that lies in the loss-free distribution of the first reaction product, i.e. of the labeled precursor compound, on the first cartridge upon transfer from the reaction vessel. When the proportion of the carrier material is too small the labeled precursor compound cannot sufficiently be collected. Therefore, amounts below 800 mg of solid carrier material are unsuitable and there are losses in the overall yield with respect to the amount of the radioactive isotope used. This, in turn results in a significant reduction of the patient doses.

Preferably, the first cartridge contains 810 to 1000 mg of the solid carrier, particularly preferred 815 to 900 mg of the solid carrier, most preferably 820 mg of the solid carrier.

If the first cartridge has the same inner diameter as the cartridge described in the embodiment of DE 697 32 599 T2, so the cartridge is longer due to the higher amounts of solid carrier.

Preferably, the reaction vessel is a reaction vessel made of a fluoride-free plastic in order to prevent exchange reactions between the fluoride of the plastic and the radio-active isotope. Preferably, the temperature-resistant plastic is a cyclic olefin copolymer. Particularly suitable are cyclic olefin copolymers having a heat deflection temperature (measured in accordance to ISO 75-1, -2 HDT/B 0.45 MPa) of 120° C. and more, in particular of 150° C. and more. A particularly suitable cyclic olefin copolymer is for example Topas®, in particular Topas® 6015S-04, of TOPAS Advanced Polymers GmbH, Frankfurt/Main, Del. It has surprisingly been found that with a reaction vessel made of cyclic olefin copolymer penetration of [¹⁸F] fluoride ions into the material of the reaction vessel is prevented and that this fact is a cause for the significant yield loss with the use of the known glass reaction vessels.

By means of the device according to the invention which comprises a first cartridge with a higher proportion of solid carrier as well as a reaction vessel made of a temperature-resistant plastic and otherwise is unchanged over DE 697 32 599 T2 a yield of at least 70% can be achieved based on the same amount of radioactive isotope used. In the prior art a yield of only 60% could be achieved. This means an increase of the doses to be achieved by approx. 17%.

In a preferred embodiment the first and the second cartridge contain the same type of solid carrier. Suitable solid carriers are for example carriers of the C18, C18 ec, C8, C4, tC18, NH₂, diol, phenyl, or polystyrene divinyl benzene type. In the case of [¹⁸F]-FDG the solid carrier preferably is C18.

Preferably, the device is automated. For that, the device should comprise valves for controlling the stream of starting materials and reaction products as well as excipients and process gases. One exemplary process gas is nitrogen (N₂), exemplary excipients are solvents such as water or acetonitrile, deprotection agents for the removal of protective groups of the precursor compound as well as fluid purifying agents. The starting materials required, the excipients, and the process gas are known to the skilled person from the prior art.

The pipelines connecting the reaction vessel, the first cartridge, and the second cartridge are tubes, for example.

Preferably, the reaction vessel, the first cartridge, the second cartridge, and the pipelines connecting the reaction vessel, the first cartridge, and the second cartridge are constituents of a disposable element. Said disposable element can be inserted into a stationary element and there, used for the one-time synthesis of the radio-labeled compound.

In one embodiment of the invention the amount of solid carrier in the first cartridge is twice or more the amount of solid carrier in the second cartridge.

In accordance to the invention further the use of the device according to the invention for the preparation of 2-desoxy-2-[⁸F]fluoro-D-glucose is provided.

Further, a method for the synthesis of radio-labeled compounds by means of the device according to the invention is provided, which comprises the following steps:
(a) reacting the precursor compound with a radioactive isotope in the reaction vessel at a temperature of 100° C. or more to obtain a first reaction product;
(b) passing the first reaction product to the first cartridge and hydrolyzing the protective groups to obtain a second reaction product; and
(c) passing the second reaction product to the second cartridge and purifying the reaction product to obtain the radio-labeled compound.

In a preferred embodiment of the method step (a) is carried out at a temperature of 120° C. or more, particularly preferred at 125° C.

The invention is explained in detail with the help of examples not intended to limit the invention with respect to the drawings. Here

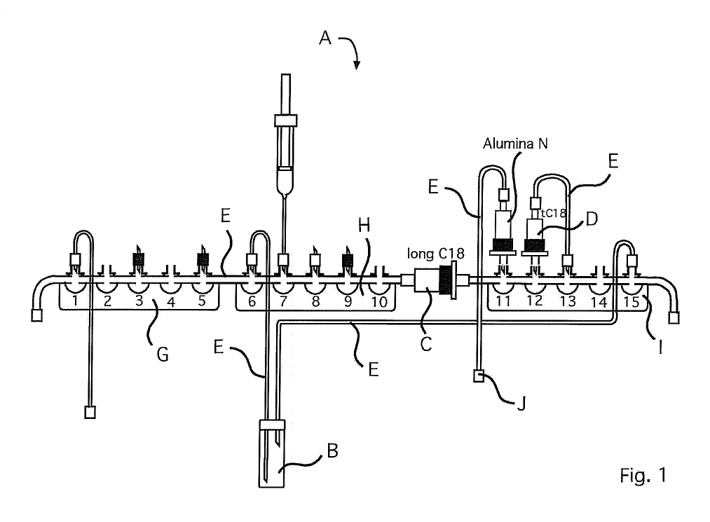

FIG. 1 shows a schematic representation of a disposable element comprising the constituents of the device according to the invention; and

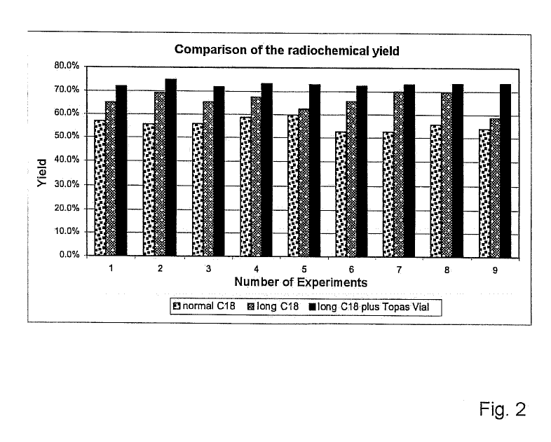

FIG. 2 shows a diagram illustrating a comparison of the radiochemical yields in the preparation of 2-desoxy-2-[¹⁸F]fluoro-D-glucose according to the prior art and according to the present invention.

### EXAMPLE 1

### Construction of One Embodiment of the Device According to the Invention

The embodiment of the device according to the invention shown in FIG. 1 comprises three tap landings G, H, I each having five control valves, wherein the tap landings G and H are connected by a tube and tap landings H and I are connected by the first cartridge, i.e. a long C18 cartridge with an increased amount of solid carrier material compared to the prior art. The reaction vessel is connected to the tap landings via tubes at control valves 6 and 15. Further, the second cartridge, a tC18 purification cartridge, is attached to control valve 12 and connected to control valve 13 via a tube. At the control valve 11 there is the exit for the final product that is finally passed over an Alumina-N cartridge for separating off excessive fluorides. Further tubes are at control valves 1 and 15 for supplying and draining off gases and liquids. At control valves 3, 5, 8, and 9 there are so-called plastic spikes onto which the storage vessels for the chemicals are fitted on. A slightly larger sized spike is connected to control valve 6 via a tube and serves for the fixation of a water reservoir for injection purposes.

### EXAMPLE 2

### Synthesis of [¹⁸F]-FDG by Means of the Device According to the Invention

The device according to the invention is inserted into the module (Tracerlab Mx of General Electric) and with the software the following operations are started:
1. Elution of the radioactive fluoride via an anion exchanger by means of a phase-transfer reagent (a mixture of Kryptofix® (K2.2.2) and potassium carbonate in water/acetonitrile).
2. Drying the fluoride activated with the phase-transfer reagent by azeotropic distillation under repeated addition of acetonitrile.
3. Reaction of the precursor compound tetra-O-acetyl-mannose Inflate (MT) with the activated fluoride at a temperature of 125° C. in the reaction vessel (Topas vial) or in a glass vial at a temperature of 105° C.
4. Dilution of the reaction mixture with water and elution over the first cartridge (C18 cartridge).
5. Hydrolysis of the acetyl protective groups on the first cartridge with 2 molar sodium hydroxide solution.
6. Elution of the target compound over the second cartridge (tC18 purification cartridge).
7. Addition of a buffer solution and elution of the target compound [¹⁸F]-FDG to the final vial via a sterile filter and an Alumina-N cartridge for separating off excessive 18F fluoride.

Apart from that, the procedure corresponds to the procedure described in DE 697 32 599 T2.

Several comparing experiments for the synthesis of [¹⁸F]-FDG have been performed, wherein the set of equipment (cassette) has the following modifications over the original in DE 697 32 599 T2:
1. no modification over DE 697 32 599 T2.
2. first C18 cartridge with 820 mg carrier material
3. first C18 cartridge with 820 mg carrier material and reaction vessel made of Topas 6015S-04

FIG. 2 shows the chronologically uncorrected radiochemical yield in the synthesis of [¹⁸F]-FDG for the respective cases with a number of nine experiments. In all experiments the change in the amount of solid carrier as well as the additional substitution of the reaction vessel result in an increase of the yield.

### LIST OF REFERENCE MARKS

A Device according to the invention
B Reaction Vessel
C First Cartridge
D Second Cartridge
E Pipelines
F Alumina-N Cartridge
G First Tap Landing
H Second Tap Landing
I Third Tap Landing
J Final Vial

1. A device for the synthesis of radio-labeled compounds, which comprises:
a reaction vessel for reacting a precursor compound having protective groups with a radioactive isotope to obtain a first reaction product;
a first cartridge for hydrolyzing the protective groups of the first reaction product to obtain a second reaction product; and
a second cartridge for purifying the second reaction product,
wherein the reaction vessel, the first cartridge, and the second cartridge are connected to each other via pipelines;
wherein,
the first cartridge contains 801 to 1200 mg of a solid carrier and the reaction vessel is a reaction vessel made of a temperature-resistant plastic with the plastic having a temperature resistance of at least 120° C.

2. The device according to claim 1,
wherein,
the first cartridge contains 810 to 1000 mg of a solid carrier.

3. The device according to claim 1,
wherein,
the first cartridge contains 815 to 900 mg of a solid carrier.

4. The device according to claim 1,
wherein,
the temperature-resistant plastic is a cyclic olefin copolymer.

5. The device according to claim 1,
wherein,
the device is automated.

6. The device according to claim 1,
wherein,
the reaction vessel, the first cartridge, the second cartridge, and the pipelines connecting the reaction vessel, the first cartridge, and the second cartridge are constituents of a disposable element.

7. The device according to claim 1,
wherein,
the device further comprises valves for controlling the stream of starting materials and reaction products as well as excipients and process gases.

8. The device according to claim 1,
wherein,
the amount of solid carrier in the first cartridge is twice or more of the amount of the solid carrier in the second cartridge.

9. (canceled)

10. A method for the synthesis of radio-labeled compounds by means of a device according to claim 1,
wherein,
it comprises:
(a) reacting the precursor compound with a radioactive isotope in the reaction vessel at a temperature of 100° C. or more to obtain a first reaction product;
(b) passing the first reaction product to the first cartridge and hydrolyzing the protective groups to obtain a second reaction product; and
(c) passing the second reaction product to the second cartridge and purifying the reaction product to obtain the radio-labeled compound.

11. The method according to claim 10,
wherein,
(a) is carried out at a temperature of 120° C. or more.
